(19) United States
(12) Patent Application Publication
(10) Pub. No.: US 2024/0350688 A1
(43) Pub. Date: Oct. 24, 2024

(54) Ultrasound Gel-Treating Stations, Systems, and Methods

(71) Applicant: Bard Access Systems, Inc., Salt Lake City, UT (US)

(72) Inventor: Matthew J. Prince, Herriman, UT (US)

(21) Appl. No.: 18/136,111

(22) Filed: Apr. 18, 2023

(51) Int. Cl. A61L 2/10; A61L 2/24; A61L 2/26

(52) U.S. Cl. CPC A61L 2/10; A61L 2/24; A61L 2/26; A61B 8/4281; A61L 2202/11; A61L 2202/23

(57) ABSTRACT

Ultrasound gel-treating stations, systems, and methods enable at least germicidal ultraviolet (“UV”)-light treatment of ultrasound gel. For example, an ultrasound gel-treating station can include a housing, a cavity within the housing, and one or more UV-light sources disposed in the cavity or the housing about the cavity. The cavity within the housing can be configured to hold one or more bottles of ultrasound gel. The one-or-more UV-light sources can be configured for irradiating the one-or-more bottles of ultrasound gel with germicidal radiation when the one-or-more bottles of ultrasound gel are disposed in the cavity. Heat dissipated by the one-or-more UV-light sources can warm the one-or-more bottles of ultrasound gel. Additionally or alternatively, the ultrasound gel-treating station can further include one or more heating elements disposed in the cavity or the housing about the cavity for warming the one-or-more bottles of ultrasound gel.

### BACKGROUND

Ultrasound gel warmers exist, but existing ultrasound gel warmers can promote bacterial growth and, therefore, be a source of infection. As such, it is not advisable to use the existing ultrasound gel warmers in sterile procedures such as those for placing percutaneous catheters. Use of packets of sterile ultrasound gel, which packets can be warmed in ultrasound gel warmers configured therefor, offer a workaround for providing warm, sterile ultrasound gel for the foregoing sterile procedures, but the packets of sterile ultrasound gel are not as economical as bottles of ultrasound gel. That, and the packets of sterile ultrasound gel produce more waste than the bottles of ultrasound gel.

Disclosed herein are ultrasound gel-treating stations, systems, and methods that address the foregoing.

### SUMMARY

Disclosed herein is an ultrasound gel-treating station including, in some embodiments, a housing, a cavity within the housing, one or more heating elements disposed in the cavity or the housing about the cavity, and one or more ultraviolet (“UV”)-light sources disposed in the cavity or the housing. The cavity within the housing is configured to hold one or more bottles of ultrasound gel. The one-or-more heating elements are configured for warming the one-or-more bottles of ultrasound gel when the one-or-more bottles of ultrasound gel are disposed in the cavity. The one-or-more UV-light sources are configured for irradiating the one-or-more bottles of ultrasound gel with germicidal radiation when the one-or-more bottles of ultrasound gel are disposed in the cavity.

In some embodiments, the ultrasound gel-treating station further includes a cover for covering the cavity when at least the one-or-more bottles of ultrasound gel are disposed in the cavity and being warmed, irradiated, or both warmed and irradiated.

In some embodiments, the ultrasound gel-treating station is configured to stop irradiating the one-or-more bottles of ultrasound gel when the cavity is uncovered.

In some embodiments, the ultrasound gel-treating station further includes a microcontroller configured for operating the ultrasound gel-treating station. The microcontroller is disposed within the housing with a sensor module including one or more sensors configured to sense conditions in the cavity for starting, stopping, or adjusting the one-or-more heating elements or the one-or-more UV-light sources.

In some embodiments, the microcontroller is further coupled to one or more visual indicators disposed in the housing for indicating when the one-or-more bottles of ultrasound gel are being warmed, irradiated, both warmed and irradiated, or subsequent thereto.

In some embodiments, the microcontroller is further coupled to a timer disposed in the housing for indicating when the one-or-more bottles of ultrasound gel will be warm, disinfected, or both warmed and disinfected.

In some embodiments, the ultrasound gel-treating station further includes an actuator disposed in the housing for lifting the one-or-more bottles of ultrasound gel at least partially out of the ultrasound gel-treating station.

In some embodiments, the ultrasound gel-treating station is integrated into a roll stand or cart.

Also disclosed herein is an ultrasound gel-treating station including, in some embodiments, a housing, a cavity within the housing, and one or more UV-light sources disposed in the cavity or the housing about the cavity. The cavity within the housing is configured to hold one or more bottles of ultrasound gel. The one-or-more UV-light sources are configured for irradiating the one-or-more bottles of ultrasound gel with germicidal radiation when the one-or-more bottles of ultrasound gel are disposed in the cavity.

In some embodiments, heat dissipated by the one-or-more UV-light sources warms the one-or-more bottles of ultrasound gel when the one-or-more bottles of ultrasound gel are disposed in the cavity.

In some embodiments, the ultrasound gel-treating station further includes a cover for covering the cavity when at least the one-or-more bottles of ultrasound gel are disposed in the cavity and being irradiated.

In some embodiments, the ultrasound gel-treating station is configured to stop irradiating the one-or-more bottles of ultrasound gel when the cavity is uncovered.

In some embodiments, the ultrasound gel-treating station further includes a microcontroller configured for operating the ultrasound gel-treating station. The microcontroller is disposed within the housing with a sensor module including one or more sensors configured to sense conditions in the cavity for starting, stopping, or adjusting the one-or-more UV-light sources.

In some embodiments, the microcontroller is further coupled to one or more visual indicators disposed in the housing for indicating when the one-or-more bottles of ultrasound gel are being irradiated.

In some embodiments, the microcontroller is further coupled to a timer disposed in the housing for indicating when the one-or-more bottles of ultrasound gel will be disinfected.

In some embodiments, the ultrasound gel-treating station further includes an actuator disposed in the housing for lifting the one-or-more bottles of ultrasound gel at least partially out of the ultrasound gel-treating station.

Also disclosed herein is an ultrasound gel-treating system including, in some embodiments, one or more bottles of ultrasound gel and an ultrasound gel-treating station. Each bottle of the one-or-more bottles is formed of a pliable polymeric material sufficiently transparent to transmit UV light therethrough. The ultrasound gel-treating station includes a housing, a cavity within the housing, and one or more UV-light sources disposed in the cavity or the housing about the cavity. The cavity within the housing is configured to hold the one-or-more bottles of ultrasound gel. The one-or-more UV-light sources are configured for irradiating the one-or-more bottles of ultrasound gel with germicidal radiation when the one-or-more bottles of ultrasound gel are disposed in the cavity.

In some embodiments, the ultrasound gel-treating station further includes one or more heating elements disposed in the cavity or the housing about the cavity for warming the one-or-more bottles of ultrasound gel when the one-or-more bottles of ultrasound gel are disposed in the cavity.

In some embodiments, heat dissipated by the one-or-more UV-light sources warms the one-or-more bottles of ultrasound gel when the one-or-more bottles of ultrasound gel are disposed in the cavity.

In some embodiments, the ultrasound gel-treating station further includes a cover for covering the cavity when at least the one-or-more bottles of ultrasound gel are disposed in the cavity and being irradiated.

In some embodiments, the ultrasound gel-treating station is configured to stop irradiating the one-or-more bottles of ultrasound gel when the cavity is uncovered.

In some embodiments, the ultrasound gel-treating station further includes a microcontroller configured for operating the ultrasound gel-treating station. The microcontroller is disposed within the housing with a sensor module including one or more sensors configured to sense conditions in the cavity for starting, stopping, or adjusting the one-or-more UV-light sources.

In some embodiments, the ultrasound gel-treating station further includes an actuator disposed in the housing for lifting the one-or-more bottles of ultrasound gel at least partially out of the ultrasound gel-treating station.

In some embodiments, the germicidal radiation is selected from broad spectrum UV-visible light, broad spectrum UV light, UVA light, UVB light, UVC light, blue light, and modulated light thereof, wherein the modulated light is modulated with respect to frequency, power, duration, or a combination thereof.

In some embodiments, the polymeric material of which the one-or-more bottles are formed is selected from a polycarbonate, a polyethylene terephthalate, a polyvinyl chloride, a polyurethane, a poly (methyl methacrylate), a polyimide, a polyetherimide, and a cyclic olefin polymer, the polymeric material optionally including one or more comonomer residues, one or more plasticizers, or a combination thereof.

These and other features of the concepts provided herein will become more apparent to those of skill in the art in view of the accompanying drawings and following description, which describe particular embodiments of such concepts in greater detail.

### DRAWINGS

FIG. 1 illustrates an ultrasound gel-treating system including an ultrasound gel-treating station and one or more bottles of ultrasound gel in accordance with some embodiments.

FIG. 2 further illustrates the ultrasound gel-treating system in accordance with some embodiments.

FIG. 3 further illustrates the ultrasound gel-treating system in accordance with some embodiments.

FIG. 4 illustrates irradiation of a bottle of ultrasound gel disposed in a cavity of the ultrasound gel-treating station in accordance with some embodiments.

FIG. 5 provides a block diagram of the ultrasound gel-treating station in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, ultrasound gel warmers exist, but existing ultrasound gel warmers can promote bacterial growth and, therefore, be a source of infection. As such, it is not advisable to use the existing ultrasound gel warmers in sterile procedures such as those for placing percutaneous catheters. Use of packets of sterile ultrasound gel, which packets can be warmed in ultrasound gel warmers configured therefor, offer a workaround for providing warm, sterile ultrasound gel for the foregoing sterile procedures, but the packets of sterile ultrasound gel are not as economical as bottles of ultrasound gel. That, and the packets of sterile ultrasound gel produce more waste than the bottles of ultrasound gel.

Disclosed herein are ultrasound gel-treating stations, systems, and methods that address the foregoing.

### Ultrasound Gel-Treating Systems

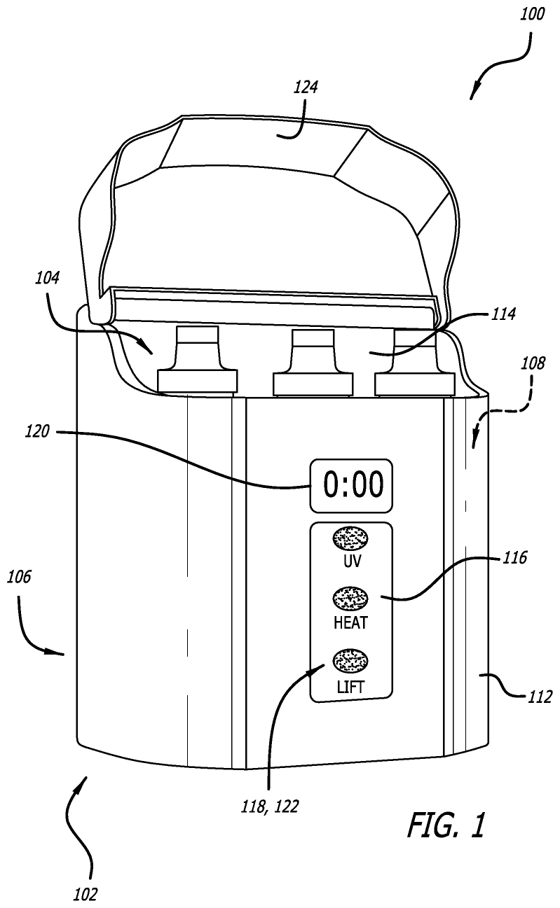

FIGS. 1-3 illustrate an ultrasound gel-treating system 100 including an ultrasound gel-treating station 102 and one or more bottles of ultrasound gel 104 in accordance with some embodiments.

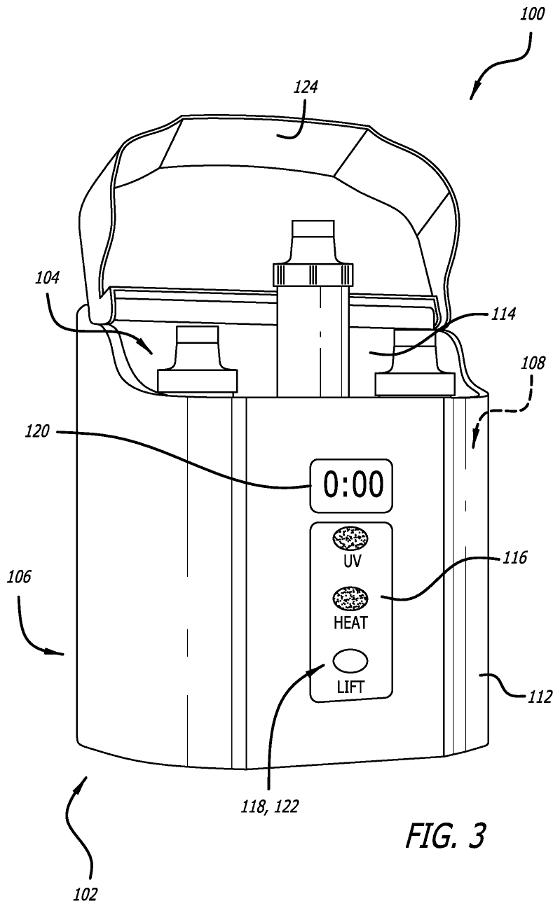

As shown in FIGS. 1 and 3, the ultrasound gel-treating system 100 can include the ultrasound gel-treating station 102, the one-or-more bottles of ultrasound gel 104, or both the ultrasound gel-treating station 102 and the one-or-more bottles of ultrasound gel 104. However, it should be understood that the ultrasound gel-treating system 100 is not limited thereto.

The ultrasound gel-treating station 102 includes a housing 106, a cavity 108 within the housing 106, and one or more UV-light sources 110 disposed in the cavity 108 or the housing 106. Notably, the ultrasound gel-treating station 102 can be a stand-alone unit in which the housing 106 can be considered its body. In other embodiments, the ultrasound gel-treating station 102 is instead integrated into a roll stand or cart.

The housing 106 can include an external housing 112 and an internal housing 114. At least a portion of the external housing 112 faces outward from the ultrasound gel-treating station 102, and at least a portion of the internal housing 114 faces inward to the ultrasound gel-treating station 102. While the external housing 112 and the internal housing 114 can be separate pieces coextensive with their namesakes, each portion of housing of the external housing 112 and the internal housing 114 can be independently formed of one or more pieces. Alternatively, the external housing 112 and the internal housing 114 are an integral piece, wherein access to an internal space between walls of the external housing 112 and the internal housing 114 is, for example, through a bottom of the housing 106.

As shown in FIG. 1, the external housing 112 can include a button interface 116, one or more visual indicators 118, a timer 120, or a combination thereof. The button interface 116 can be disposed in the external housing 112, and the button interface 116 can include one or more buttons 122. The one-or-more visual indicators 118 can be integrated into the one-or-more buttons 122, as shown, or the one-or-more visual indicators 118 can be disposed in the external housing 112 separate from the one-or-more buttons 122. The timer 120 can be disposed in the external housing 112 proximal the button interface 116 such as above or below the button interface 116, but the timer 120 need not be limited thereto.

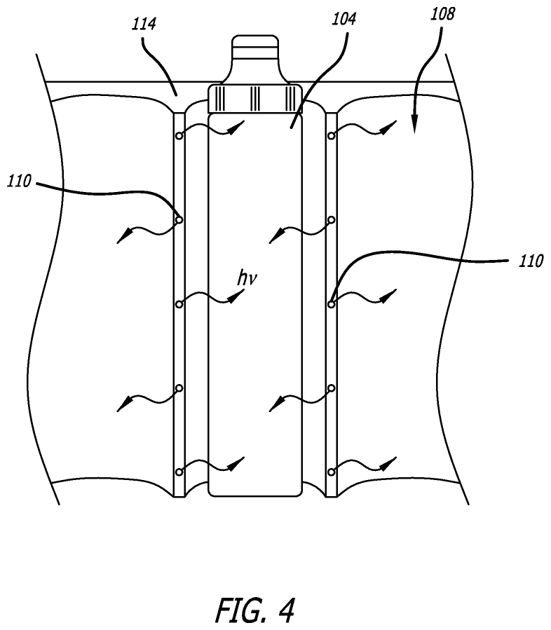

The cavity 108 within the housing 106, specifically, within the internal housing 114, can be configured to hold the one-or-more bottles of ultrasound gel 104. Indeed, as shown in FIG. 4, the cavity 108 can include a plurality of sleeves or silos formed from the internal housing 114 into which a plurality of the bottles of ultrasound gel 104 can be inserted. Such sleeves or silos can be advantageous in that any bottle of the plurality of bottles of ultrasound gel 104 used in a procedure can be returned to its sleeve or silo after the procedure without contaminating an adjacent bottle of the plurality of bottles of ultrasound gel 104.

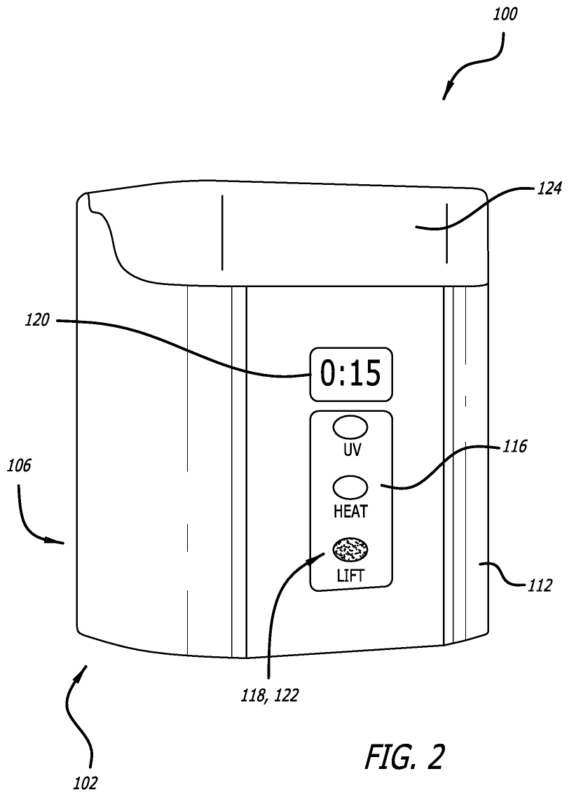

As shown in FIGS. 1 and 2, the ultrasound gel-treating station 102 can include a cover 124 for covering the cavity 108 when at least the one-or-more bottles of ultrasound gel 104 are disposed in the cavity 108 and being treated. Such a cover 124 can be a hinged lid, as shown, a retractable cover (e.g., a tambour door), a mechanical diaphragm (e.g., mechanical iris), or the like for covering the cavity 108 when at least the one-or-more bottles of ultrasound gel 104 are disposed in the cavity 108 and being irradiated, warmed, or both irradiated and warmed. Notably, for user protection, the ultrasound gel-treating station 102 is configured to stop irradiating the one-or-more bottles of ultrasound gel 104 or the cavity 108, itself, when the cavity 108 is uncovered such as by lifting the hinged lid, retracting the retractable cover, opening the mechanical diaphragm, or the like. However, the ultrasound gel-treating station 102 need not stop warming the one-or-more bottles of ultrasound gel 104 or the cavity 108, itself, when the cavity 108 is uncovered; that is, unless the warming is dependent upon the heat dissipated by the one-or-more UV-light sources 110.

FIG. 4 illustrates irradiation of a bottle of ultrasound gel disposed in the cavity 108 of the ultrasound gel-treating station 102 in accordance with some embodiments.

As shown, the one-or-more UV-light sources 110 can be disposed in the cavity 108, in the housing 106, behind the housing 106, or the like, optionally, in a combination thereof, for irradiating the one-or-more bottles of ultrasound gel 104 with germicidal radiation when the one-or-more bottles of ultrasound gel 104 are disposed in the cavity 108. When the one-or-more UV-light sources 110 are disposed in the cavity 108, the one-or-more UV-light sources 110 can be wholly disposed in the cavity 108 with corresponding electrical leads passing through through holes in the internal housing 114 as well as the internal space between the walls of the external and internal housing 114 to the microcontroller 128. When the one-or-more UV-light sources 110 are disposed in the housing 106, the one-or-more UV-light sources 110 can be partially disposed in the cavity 108 (e.g., peaking into the cavity 108) with a remainder of the one-or-more UV-light sources 110 disposed in the through holes of the internal housing 114, the corresponding electrical leads passing through the internal space between the walls of the external and internal housing 112 and 114 to the microcontroller 128. Lastly, when the one-or-more UV-light sources 110 are behind the housing 106, the one-or-more UV-light sources 110 can be mounted in the internal space between the walls of the external and internal housing 112 and 114 such that the one-or-more UV-light sources 110 emit the germicidal radiation toward the cavity 108, at least the internal housing 114 around the cavity 108 being a polymeric material sufficiently transparent to transmit UV light therethrough, optionally, a rigid formulation of the pliable polymeric material set forth below. As in other embodiments, the corresponding electrical leads of the one-or-more UV-light sources 110 pass through the internal space between the walls of the external and internal housing 112 and 114 to the microcontroller 128.

The one-or-more UV-light sources 110 can be independently selected from a low-pressure mercury lamp, an excimer lamp, a pulsed xenon lamp, and a semiconductor light source, the semiconductor light source, in turn, selected from a light-emitting diode (“LED”), a laser, and a superluminescent diode (“SLD”), which SLD, notably, combines high-power and brightness characteristics of lasers with low-power characteristic of LEDs. By way of example, FIG. 4 shows a plurality of LEDs disposed in the internal housing 114 and emitting the germicidal radiation into the cavity 108. Such germicidal radiation emitted by the one-or-more UV-light sources 110 can be selected from broad spectrum UV-visible light, broad spectrum UV light, UVA light, UVB light, UVC light, blue light, and modulated light thereof, wherein the modulated light is modulated with respect to wavelength or frequency, power, including ramping the power, duration, including pulse durations when pulsing the modulated light, or a combination thereof.

Notably, heat dissipated by the one-or-more UV-light sources 110 can warm the one-or-more bottles of ultrasound gel 104 when the one-or-more bottles of ultrasound gel 104 are disposed in the cavity 108. Notwithstanding the foregoing, the ultrasound gel-treating station 102 can further include one or more heating elements 126 for warming the one-or-more bottles of ultrasound gel 104 when the one-or-more bottles of ultrasound gel 104 are disposed in the cavity 108.

The one-or-more heating elements 126 can be disposed in the cavity 108, in the housing 106, behind the housing 106, or the like, optionally, in a combination thereof, for warming the one-or-more bottles of ultrasound gel 104 when the one-or-more bottles of ultrasound gel 104 are disposed in the cavity 108. When the one-or-more heating elements 126 are disposed in the cavity 108, the one-or-more heating elements 126 can be wholly disposed in the cavity 108 with corresponding electrical leads passing through through holes in the internal housing 114 as well as the internal space between the walls of the external and internal housing 112 and 114 to the microcontroller 128. When the one-or-more heating elements 126 are disposed in the housing 106, the one-or-more heating elements 126 can be partially disposed in the cavity 108 (e.g., peaking into the cavity 108) with a remainder of the one-or-more heating elements 126 disposed in the through holes of the internal housing 114, the corresponding electrical leads passing through the internal space between the walls of the external and internal housing 112 and 114 to the microcontroller 128. Lastly, when the one-or-more heating elements 126 are behind the housing 106, the one-or-more heating elements 126 can be mounted in the internal space between the walls of the external and internal housing 112 and 114 such that the one-or-more heating elements 126 emit infrared radiation toward the cavity 108. As in other embodiments, the corresponding electrical leads of the one-or-more heating elements 126 pass through the internal space between the walls of the external and internal housing 112 and 114 to the microcontroller 128.

Notably, when both the one-or-more UV-light sources 110 and the one-or-more heating elements 126 are present, the one-or-more UV-light sources 110 and the one-or-more heating elements 126 can independently adopt any configuration set forth above with respect to being disposed in the cavity 108, in the housing 106, behind the housing 106, or the like.

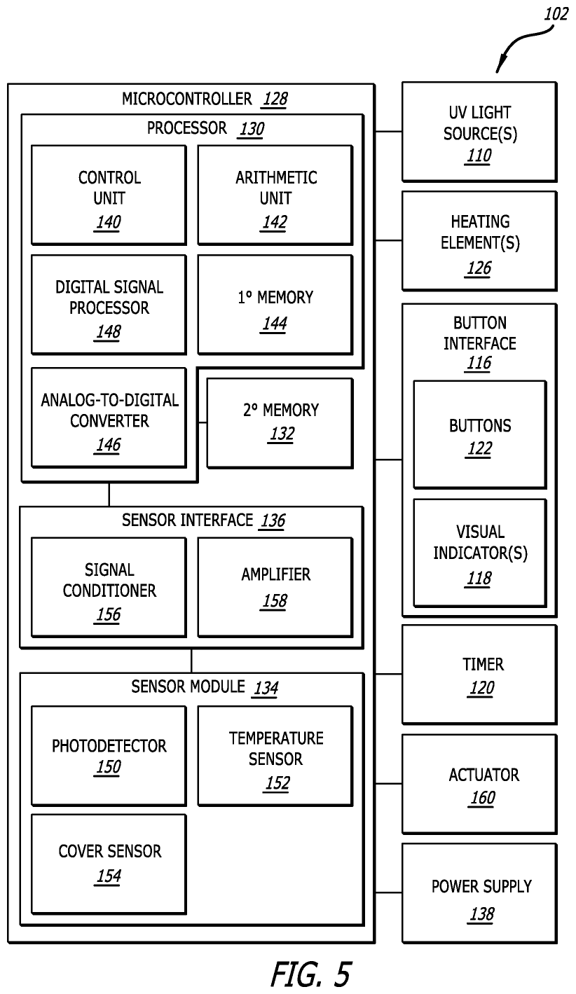

FIG. 5 provides a block diagram of the ultrasound gel-treating station 102 including a microcontroller 128 in accordance with some embodiments.

As shown, the ultrasound gel-treating station 102 can further include the microcontroller 128 configured for operating the ultrasound gel-treating station 102. The microcontroller 128 can be disposed within the housing 106 such as the internal space between the walls of the external and internal housing 112 and 114.

The microcontroller 128 can include components selected from at least a processor 130, secondary memory 132, a sensor module 134, a sensor interface 136, and a power supply 138 such as an internal power supply (e.g., a battery) or an external power supply (e.g., utility power). The processor 130 can include a control unit 140, an arithmetic unit 142, and primary memory 144 (e.g., cache memory, RAM, or both), wherein the primary memory 144 can be configured to store in-use programs and data (e.g., the sensor data). While the primary memory 144 can be within the same package as a remainder of the processor 130 as alluded to in FIG. 5, at least the foregoing RAM can be distributed outside the package of the processor 130, for example, in its own package.

The secondary memory 132 can be configured to store data and programs including instructions, logic, algorithms including machine-learning algorithms, artificial intelligence (“AI”) models, or some combination thereof for loading into the primary memory 144 for use by the processor 130, for example, when determining from, for example, the sensor data, whether the one-or-more bottles of ultrasound gel 104 need to be disinfected, warmed, or both disinfected and warmed, whether the one-or-more bottles of ultrasound gel 104 are being irradiated, warmed, or both irradiated and warmed, how much longer the one-or-more bottles of ultrasound gel 104 should be irradiated, warmed, or both irradiated and warmed, whether the one-or-more bottles of ultrasound gel 104 have been disinfected, warmed, or both disinfected and warmed, or some combination thereof. Should the microcontroller 128 include the sensor module 134 and the sensor interface 136 to sense conditions in the cavity 108 for starting, stopping, or adjusting the one-or-more UV-light sources 110, the one-or-more heating elements 126, or both the one-or-more UV-light sources 110 and the one-or-more heating elements 126 in accordance with the foregoing, the processor 130 can further include an analog-to-digital converter 146 (“ADC”) configured to convert electrical signals from the one-or-more sensors from analog to digital and a digital-signal processor 148 (“DSP”) configured to generate sensor data from the electrical signals. While the ADC and DSP 146 and 148 can be within the same package as a remainder of the processor 130 as alluded to in FIG. 5, the ADC and DSP 146 and 148 can be distributed outside the package of the processor 130, for example, in their own package.

When present, the sensor module 134 can include one or more sensors selected from at least a photodetector 150, a temperature sensor 152, and a cover sensor 154 configured to generate electrical signals in response to photons detected, temperature sensed, and cover-state sensed, respectively, wherein the cover-state sensed can be, for example, open cover, ajar cover, or closed cover. Further, when present, the sensor interface 136 can include a signal conditioner 156 configured to standardize the electrical signals through voltage or current limiting, anti-aliasing filtering, or the like. In addition, the sensor interface 136 can include an amplifier 158 configured to amplify the electrical signals and, thereby, increase their signal-to-noise ratio. Again, the ADC 146 can be configured to convert the electrical signals from analog to digital, and the DSP 148 can be configured to generate the sensor data from the electrical signals for determining whether to start, stop, or adjust the one-or-more UV-light sources 110, the one-or-more heating elements 126, or both the one-or-more UV-light sources 110 and the one-or-more heating elements 126 in accordance with the sensor data.

The microcontroller 128 can be coupled to the one-or-more visual indicators 118 disposed in the housing 106 for indicating the one or more bottle of ultrasound gel need to be disinfected, warmed, or both disinfected and warmed, the one-or-more bottles of ultrasound gel 104 are being irradiated, warmed, or both irradiated and warmed, or the one-or-more bottles of ultrasound gel 104 have been disinfected, warmed, or both disinfected and warmed. Indeed, the one-or-more visual indicators 118 can be integrated into the one-or-more buttons 122 as LEDs behind the one-or-more buttons 122, the LEDs having different colors (e.g., red, yellow, and green) of light to indicate the one or more bottle of ultrasound gel need to be disinfected, warmed, or both disinfected and warmed (e.g., red light), the one-or-more bottles of ultrasound gel 104 are being irradiated, warmed, or both irradiated and warmed (e.g., yellow light), or the one-or-more bottles of ultrasound gel 104 have been disinfected, warmed, or both disinfected and warmed (e.g., green light). Additionally or alternatively, the microcontroller 128 can be coupled to the timer 120 disposed in the external housing 112 for indicating, for example, by a countdown, when the one-or-more bottles of ultrasound gel 104 will be disinfected, warmed, or both disinfected and warmed.

The ultrasound gel-treating station 102 can further include an actuator 160 disposed in the housing 106 for lifting the one-or-more bottles of ultrasound gel 104 at least partially out of the ultrasound gel-treating station 102 so any bottle of the one-or-more bottles of ultrasound gel 104 can be grabbed without touching a potentially contaminated exterior surface of the ultrasound gel-treating station 102. The actuator 160 can be coupled to one or more movable bottoms of the plurality of sleeves or silos of the internal housing 114 set forth above for lifting the one-or-more bottles of ultrasound gel 104 at least partially out of the ultrasound gel-treating station 102.

As to the one-or-more bottles of ultrasound gel 104, each bottle of the one-or-more bottles of ultrasound gel 104 can be formed of a pliable polymeric material sufficiently transparent to transmit UV light therethrough. Such a polymeric material can be selected from a polycarbonate, a polyethylene terephthalate, a polyvinyl chloride, a polyurethane, a poly (methyl methacrylate), a polyimide, a polyetherimide, and a cyclic olefin polymer, the polymeric material optionally including one or more comonomer residues, thereby making the foregoing polymeric material a copolymer, one or more plasticizers, or a combination thereof.

### Methods

Methods include methods of the ultrasound gel-treating system or station 100 or 102, itself, as well as methods of using the ultrasound gel-treating system 100.

A method of the ultrasound gel-treating station 102 can include at least a sensing operation of sensing conditions in the cavity 108 and a determining operation of determining whether the one-or-more UV-light sources 110, the one-or-more heating elements 126, or both the one-or-more UV-light sources 110 and the one-or-more heating elements 126 should be started, stopped, or adjusted. Further, the method of the ultrasound gel-treating station 102 can include an indicating operation of indicating with the one-or-more visual indicators 118 the one-or-more bottles of ultrasound gel 104 need to be disinfected, warmed, or both disinfected and warmed, the one-or-more bottles of ultrasound gel 104 are being irradiated, warmed, or both irradiated and warmed, or the one-or-more bottles of ultrasound gel 104 have been disinfected, warmed, or both disinfected and warmed. Such operations of the ultrasound gel-treating station 102 and others can be further understood from disclosure set forth above.

A method of using the ultrasound gel-treating station 102 can include at least a witnessing operation of witnessing the ultrasound gel-treating station 102 indicating with the one-or-more visual indicators 118 that the plurality of bottles of ultrasound gel 104 have been disinfected, warmed, or both disinfected and warmed. Further, the method of using the ultrasound gel-treating station 102 can include an uncovering operation of uncovering the cavity 108, a waiting operation of waiting for the actuator 160 to lift the plurality of bottles of ultrasound gel 104 at least partially out of the ultrasound gel-treating station 102, and a grabbing operation of grabbing a bottle of the plurality of bottles of ultrasound gel 104 without touching a potentially contaminated exterior surface of the ultrasound gel-treating station 102. Further yet, the method of using the ultrasound gel-treating station 102 can include returning the bottle to its sleeve or silo without contaminating any other bottle of the plurality of bottles of ultrasound gel 104.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An ultrasound gel-treating station, comprising:
a housing;
a cavity within the housing configured to hold one or more bottles of ultrasound gel;
one or more heating elements disposed in the cavity or the housing about the cavity for warming the one-or-more bottles of ultrasound gel when the one-or-more bottles of ultrasound gel are disposed in the cavity; and
one or more ultraviolet (“UV”)-light sources disposed in the cavity or the housing about the cavity for irradiating the one-or-more bottles of ultrasound gel with germicidal radiation when the one-or-more bottles of ultrasound gel are disposed in the cavity.

2. The ultrasound gel-treating station of claim 1, further comprising a cover for covering the cavity when at least the one-or-more bottles of ultrasound gel are disposed in the cavity and being warmed, irradiated, or both warmed and irradiated.

3. The ultrasound gel-treating station of claim 2, wherein the ultrasound gel-treating station is configured to stop irradiating the one-or-more bottles of ultrasound gel when the cavity is uncovered.

4. The ultrasound gel-treating station of claim 1, further comprising a microcontroller configured for operating the ultrasound gel-treating station, the microcontroller disposed within the housing with a sensor module including one or more sensors configured to sense conditions in the cavity for starting, stopping, or adjusting the one-or-more heating elements or the one-or-more UV-light sources.

5. The ultrasound gel-treating station of claim 4, wherein the microcontroller is further coupled to one or more visual indicators disposed in the housing for indicating when the one-or-more bottles of ultrasound gel are being warmed, irradiated, both warmed and irradiated, or subsequent thereto.

6. The ultrasound gel-treating station of claim 4, wherein the microcontroller is further coupled to a timer disposed in the housing for indicating when the one-or-more bottles of ultrasound gel will be warm, disinfected, or both warmed and disinfected.

7. The ultrasound gel-treating station of claim 1, further comprising an actuator disposed in the housing for lifting the one-or-more bottles of ultrasound gel at least partially out of the ultrasound gel-treating station.

8. The ultrasound gel-treating station of claim 1, wherein the ultrasound gel-treating station is integrated into a roll stand or cart.

9. An ultrasound gel-treating station, comprising:
a housing;
a cavity within the housing configured to hold the one-or-more bottles of ultrasound gel;
one or more ultraviolet (“UV”)-light sources disposed in the cavity or the housing about the cavity for irradiating the one-or-more bottles of ultrasound gel with germicidal radiation when the one-or-more bottles of ultrasound gel are disposed in the cavity.

10. The ultrasound gel-treating station of claim 9, wherein heat dissipated by the one-or-more UV-light sources warms the one-or-more bottles of ultrasound gel when the one-or-more bottles of ultrasound gel are disposed in the cavity.

11. The ultrasound gel-treating station of claim 9, further comprising a cover for covering the cavity when at least the one-or-more bottles of ultrasound gel are disposed in the cavity and being irradiated.

12. The ultrasound gel-treating station of claim 11, wherein the ultrasound gel-treating station is configured to stop irradiating the one-or-more bottles of ultrasound gel when the cavity is uncovered.

13. The ultrasound gel-treating station of claim 9, further comprising a microcontroller configured for operating the ultrasound gel-treating station, the microcontroller disposed within the housing with a sensor module including one or more sensors configured to sense conditions in the cavity for starting, stopping, or adjusting the one-or-more UV-light sources.

14. The ultrasound gel-treating station of claim 13, wherein the microcontroller is further coupled to one or more visual indicators disposed in the housing for indicating when the one-or-more bottles of ultrasound gel are being irradiated.

15. The ultrasound gel-treating station of claim 13, wherein the microcontroller is further coupled to a timer disposed in the housing for indicating when the one-or-more bottles of ultrasound gel will be disinfected.

16. The ultrasound gel-treating station of claim 9, further comprising an actuator disposed in the housing for lifting the one-or-more bottles of ultrasound gel at least partially out of the ultrasound gel-treating station.

17. An ultrasound gel-treating system, comprising:
one or more bottles of ultrasound gel, each bottle of the one-or-more bottles formed of a pliable polymeric material sufficiently transparent to transmit ultraviolet (“UV”) light therethrough;
an ultrasound gel-treating station including:
a housing;
a cavity within the housing configured to hold the one-or-more bottles of ultrasound gel;
one or more ultraviolet (“UV”)-light sources disposed in the cavity or the housing about the cavity for irradiating the one-or-more bottles of ultrasound gel with germicidal radiation when the one-or-more bottles of ultrasound gel are disposed in the cavity.

18. The ultrasound gel-treating system of claim 17, the ultrasound gel-treating station further comprising one or more heating elements disposed in the cavity or the housing about the cavity for warming the one-or-more bottles of ultrasound gel when the one-or-more bottles of ultrasound gel are disposed in the cavity.

19. The ultrasound gel-treating system of claim 17, wherein heat dissipated by the one-or-more UV-light sources warms the one-or-more bottles of ultrasound gel when the one-or-more bottles of ultrasound gel are disposed in the cavity.

20. The ultrasound gel-treating system of claim 17, the ultrasound gel-treating station further comprising a cover for covering the cavity when at least the one-or-more bottles of ultrasound gel are disposed in the cavity and being irradiated.

21. The ultrasound gel-treating system of claim 17, wherein the ultrasound gel-treating station is configured to stop irradiating the one-or-more bottles of ultrasound gel when the cavity is uncovered.

22. The ultrasound gel-treating system of claim 17, the ultrasound gel-treating station further comprising a microcontroller configured for operating the ultrasound gel-treating station, the microcontroller disposed within the housing with a sensor module including one or more sensors configured to sense conditions in the cavity for starting, stopping, or adjusting the one-or-more UV-light sources.

23. The ultrasound gel-treating system of claim 17, the ultrasound gel-treating station further comprising an actuator disposed in the housing for lifting the one-or-more bottles of ultrasound gel at least partially out of the ultrasound gel-treating station.

24. The ultrasound gel-treating system of claim 17, wherein the germicidal radiation is selected from broad spectrum UV-visible light, broad spectrum UV light, UVA light, UVB light, UVC light, blue light, and modulated light thereof, wherein the modulated light is modulated with respect to frequency, power, duration, or a combination thereof.

25. The ultrasound gel-treating system of claim 17, wherein the polymeric material of which the one-or-more bottles are formed is selected from a polycarbonate, a polyethylene terephthalate, a polyvinyl chloride, a polyurethane, a poly (methyl methacrylate), a polyimide, a polyetherimide, and a cyclic olefin polymer, the polymeric material optionally including one or more comonomer residues, one or more plasticizers, or a combination thereof.
